(19) United States
(12) Patent Application Publication
(10) Pub. No.: US 2023/0157808 A1
(43) Pub. Date: May 25, 2023

(54) REINFORCED KNEE METHOD AND APPARATUS

(71) Applicant: Little Engine, LLC, Belmont, NC (US)

(72) Inventors: J. Dean Cole, Orlando, FL (US); Franz W. Kellar, Gastonia, NC (US); Michael D. Bissette, Belmont, NC (US); Harold L. Crowder, Concord, NC (US)

(21) Appl. No.: 17/970,851

(22) Filed: Oct. 21, 2022

Related U.S. Application Data
(62) Division of application No. 17/533,367, filed on Nov. 23, 2021, now abandoned.

(51) Int. Cl. A61F 2/08; A61B 17/04

(52) U.S. Cl. CPC A61F 2/0811; A61B 17/0401; A61F 2/0805; A61B 2017/0464; A61B 2017/0445; A61B 17/1675

(57) ABSTRACT

An anchor for anchoring tensile members to bone includes: a housing having a hollow interior; a collet in the interior of the housing, having a central bore for accepting tensile members therethrough and an exterior surface, wherein the collet is configured to be swaged around the tensile members; a sleeve having opposed interior and exterior surfaces, the sleeve disposed in the hollow interior of the housing and positioned adjacent to the collet, so as to be movable between first and second positions; wherein at least one of the exterior surface of the collet and the interior surface of the sleeve is tapered and the sleeve and the collet are arranged such that movement of the sleeve from the first position to the second position will cause the collet to swage radially inwards around the tensile members; and a flange element, wherein the housing is pivotally mounted to the flange element.

### BACKGROUND OF THE INVENTION

This invention relates generally to medical implants, and more particularly to a method for treating an injured, diseased, or worn human knee joint.

### BACKGROUND

Total knee arthroplasty (“TKA”) is a procedure for treating an injured, diseased, or worn human knee joint. In a TKA, an endoprosthetic joint is implanted, replacing the bearing surfaces of the joint with artificial members. Proper alignment of the joint and substantially equal tension in the soft tissues surrounding the joint are important factors in producing a good surgical outcome.

A human knee joint “J” is shown in FIGS. 1-4. The joint J is prepared for implantation by cutting away portions of the femur “F” and the tibia “T”. FIGS. 1 and 2 show the joint in extension, with cutting planes for a tibial cut 1 and a distal femoral cut 2. The tibial cut 1 and the distal formal cut 2 cooperate to define an extension gap “EG”. FIGS. 3 and 4 show the joint J in flexion, with cutting plane 3 for a posterior cut. The tibial cut 1 and the posterior cut 3 cooperate to define a flexion gap “FG”.

A goal of total knee arthroplasty is to obtain symmetric and balanced flexion and extension gaps FG, EG (in other words, two congruent rectangles). These gaps are generally measured in millimeters of separation, are further characterized by a varus or valgus angle measured in degrees, and are measured after the tibia cut, distal femoral cut, and posterior femoral cut have been done (to create flat surfaces from which to measure). It follows that, to achieve this balance, the ligament tension in the lateral and medial ligaments would be substantially equal on each side, and in each position; it also follows that the varus/valgus angle in flexion and extension would be 0°.

One problem with prior art TKA techniques is that they often irreversibly sacrifice the posterior cruciate ligament (“PCL”). These existing techniques fail to take into account the native knee kinematics controlled by the PCL and offer no way to replicate or control these kinematics post-operatively. Even techniques that intend to preserve the PCL pose a risk of damaging the ligament, in which case there is no means of restoring its strength and function during the arthroplasty procedure. In the prior art, producing a posterior-stabilized knee takes additional time due to the extra steps required to remove the PCL.

### BRIEF SUMMARY OF THE INVENTION

This problem is addressed by a method and apparatus for reinforcing the cruciate ligament structure of a knee.

According to one aspect of the technology described herein, an anchor for anchoring one or more tensile members to bone, includes: a housing extending along a central axis between open first and second ends, and having a hollow interior; a collet disposed in the hollow interior of the housing, the collet having a peripheral wall defining a central bore for accepting one or more tensile members therethrough and an exterior surface, wherein the collet is configured to be swaged around and against the one or more tensile members; a sleeve having a peripheral wall defining opposed interior and exterior surfaces, the sleeve disposed in the hollow interior of the housing and positioned generally axially adjacent to the collet, so as to be movable parallel to the central axis between first and second positions; wherein at least one of the exterior surface of the collet and the interior surface of the sleeve is tapered and the sleeve and the collet are arranged such that movement of the sleeve from the first position to the second position will cause the interior surface of the sleeve to bear against the exterior surface of the collet, causing the collet to swage radially inwards around and against the one or more tensile members; and a flange element, wherein the housing is pivotally mounted to the flange element.

### BRIEF DESCRIPTION OF THE DRAWINGS

The invention may be best understood by reference to the following description taken in conjunction with the accompanying drawing figures in which:

FIG. 1 is a view of the anterior aspect of the human knee joint in extension showing cutting planes for a total knee arthroplasty;

FIG. 2 is a view of the lateral aspect of the human knee joint of FIG. 1;

FIG. 3 is a view of the anterior aspect of the human knee joint in flexion showing cutting planes for a total knee arthroplasty;

FIG. 4 is a view of the lateral aspect of the human knee joint of FIG. 3;

FIG. 5 is an exploded perspective view of the posterior aspect of a cruciate-retaining (“CR”) knee implant;

FIG. 6 shows the knee implant of FIG. 5 in an assembled condition;

FIG. 7 illustrates a short segment of a representative tensile member;

FIGS. 8, 9, 10, and 11 are schematic views of the anterior, lateral-posterior, medial, and posterior aspects, respectively of a human knee joint, showing a total knee replacement in conjunction with a double strand posterior cruciate ligament reinforcement;

FIG. 12 is a schematic view of the posterior-lateral aspect of a human knee joint, showing a total knee replacement in conjunction with a double strand posterior cruciate ligament reinforcement;

FIG. 13 is a schematic view of the posterior aspect of a human knee joint, showing a total knee replacement in conjunction with a double strand posterior cruciate ligament reinforcement;

FIGS. 14 and 15 are schematic views of the lateral-posterior and posterior aspects, respectively of a human knee joint, showing a total knee replacement in conjunction with a double strand posterior cruciate ligament reinforcement, showing intraosseous articular points;

FIGS. 16, 17, 18, and 19 are schematic views of the anterior, lateral-posterior, medial, and posterior aspects, respectively of a human knee joint, showing a total knee replacement in conjunction with a double strand posterior cruciate ligament reinforcement;

FIGS. 20-22 are diagrammatic views of a human knee joint in extended, mid-flexed, and extended positions respectively;

FIGS. 23-25 are diagrammatic views of a human knee joint in extended, mid-flexed, and extended positions respectively;

FIG. 26 is a view of the lateral aspect of a human knee joint, showing a total knee replacement;

FIG. 27 is a view of tibia having a tibial tray implanted therein;

FIGS. 28, 29, and 30 are schematic views of the anterior, lateral-medial, and posterior aspects, respectively of a human knee joint having a tibial tray implanted thereon, with a drill guide coupled to the tibial tray;

FIG. 31 is a view of the drill guide of FIG. 28 incorporating a drill stop;

FIG. 32 is a schematic view of the anterior-lateral aspect of a human knee joint having a tibial tray implanted thereon, with a drill guide coupled to the tibial tray, along with a curved drill;

FIG. 33 is a view of the human knee joint of FIG. 32, with a curved drill inserted into the drill guide;

FIG. 34 is a schematic perspective view of an adjustable drill guide;

FIGS. 35, 36, and 37 are schematic views of the anterior, inferior, and posterior aspects, respectively of a human femur, with a drill guide coupled to the femur;

FIGS. 38, 39, and 40 are schematic views of the anterior, inferior, and posterior aspects, respectively of a human femur, with an alternative drill guide coupled to the femur;

FIGS. 41 and 42 are schematic views of the anterior, and posterior aspects, respectively of a human knee joint having two uni-compartmental protheses implanted therein;

FIG. 43 is a front elevation view of an exemplary snap-off anchor;

FIG. 44 is a side elevation view of the snap-off anchor of FIG. 43;

FIG. 45 is a cross-sectional view of the snap-off anchor of FIG. 43;

FIG. 46 is a perspective view of the snap-off anchor of FIG. 43, in a snapped-off condition;

FIG. 47 is a cross-sectional view of the snap-off anchor of FIG. 43;

FIG. 48 is a perspective view of an exemplary T-pivot anchor;

FIG. 49 is a cross-sectional view of the T-pivot anchor of FIG. 47;

FIG. 50 is a perspective view of the T-pivot anchor of FIG. 47, coupled to an insertion instrument;

FIG. 51 is another perspective view of the T-pivot anchor of FIG. 47;

FIG. 52 is an exploded perspective view of the T-pivot anchor of FIG. 47;

FIG. 53 is a top plan view of the T-pivot anchor of FIG. 42;

FIG. 54 is a side elevation view of the T-pivot anchor of FIG. 42;

FIG. 55 is a perspective view of an anchor implanted into bone;

FIG. 56 is a cross-sectional view of the anchor of FIG. 55 at a first angle;

FIG. 57 is a cross-sectional view of the anchor of FIG. 55 at a second angle;

FIG. 58 is a partially-sectioned perspective view of the anchor of FIG. 55;

FIG. 59 is another perspective view of the anchor of FIG. 55;

FIG. 60 is a side view of an anchor;

FIG. 61 is another side view of the anchor of FIG. 60;

FIG. 62 is a side view of a threaded anchor;

FIG. 63 is a schematic view of the anterior aspect of a human knee joint having a tibial tray implanted thereon, with an insertion instrument being used to tension an anchor; and

FIG. 64 is a schematic perspective view of a human knee joint with sleeves protecting tensile members implanted therein.

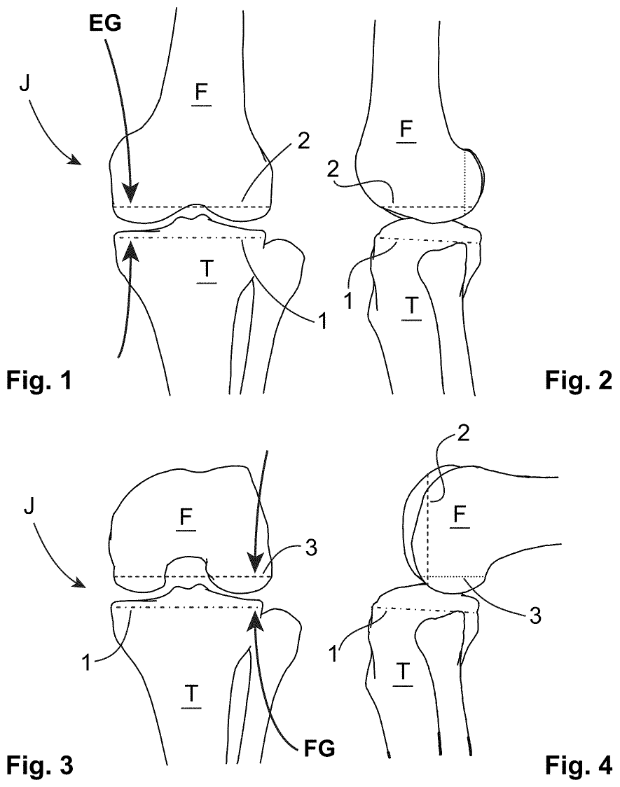

### DETAILED DESCRIPTION OF THE INVENTION

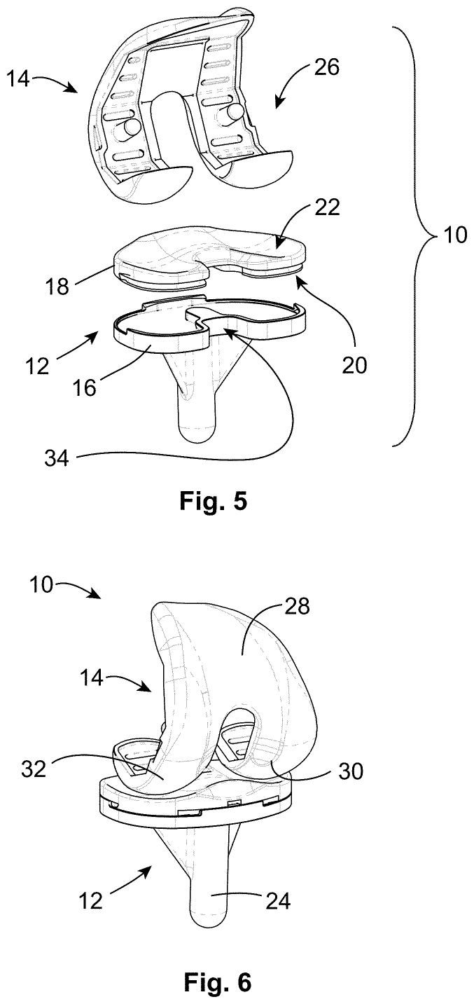

FIGS. 5 and 6 depict an exemplary endoprosthetic 10 (i.e., implant) of a known type. The endoprosthetic 10 includes a tibial component 12 and a femoral component 14. The tibial component 12 is made up of a tibial tray 16 and an insert 18. The insert 18 has a back surface 20 which abuts the tibial tray 16 and an opposed articular surface 22. The tray includes a prominent keel 24 protruding in the inferior direction (i.e. down a longitudinal axis of the tibia). The tibial tray 16 may be made from a hard, wear-resistant material such as a biocompatible metal alloy. The insert 18 may be made from a low-friction material such as a biocompatible plastic.

The femoral component 14 includes a back surface 26 shaped to abut a surface of the femur that has been appropriately shaped and an articular surface 28 comprising medial and lateral condyles 30 and 32, respectively. The femoral component 14 may be made from a hard, wear-resistant material such as a biocompatible metal alloy.

The tibial tray 16 is implanted into the tibia T and the femoral component 14 is implanted into the femur F. The insert 18 is placed into the tibial tray 16. The articular surface 22 of the insert 18 bears against the articular surface 28 of the femoral component 14, defining a functional joint.

In the illustrated example, the implant 10 is of the cruciate-retaining (“CR”) type. It includes a cutout or notch 34 in the posterior aspect of the tibial component 12 which provides a space for the posterior cruciate ligament (“PCL”).

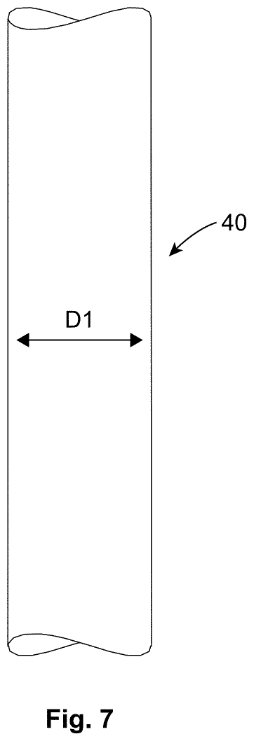

In addition to retaining the patients' PCL in a knee arthroplasty, it may be augmented (reinforced) using one or more artificial tensile members. The term “tensile member” as used herein generally refers to any flexible element capable of transmitting a tensile force. Nonlimiting examples of known types of tensile members include sutures and orthopedic cables. FIG. 7 illustrates a short segment of a representative tensile member 40 having a diameter “D1”. Commercially-available tensile members intended to be implanted in the human body may have a diameter “D 1” ranging from tens of microns in diameter to multiple millimeters in diameter. Commercially-available tensile members may be made from a variety of materials such as polymers or metal alloys. Nonlimiting examples of suitable materials include absorbable polymers, nylon, ultrahigh molecular weight polyethylene (“UBMWPE”) or polypropylene titanium alloys, or stainless steel alloys. Known physical configurations of tensile members include monofilament, braided, twisted, woven, and wrapped. Optionally, the tensile member may be made from a shape memory material, such as a temperature-responsive or moisture-response material.

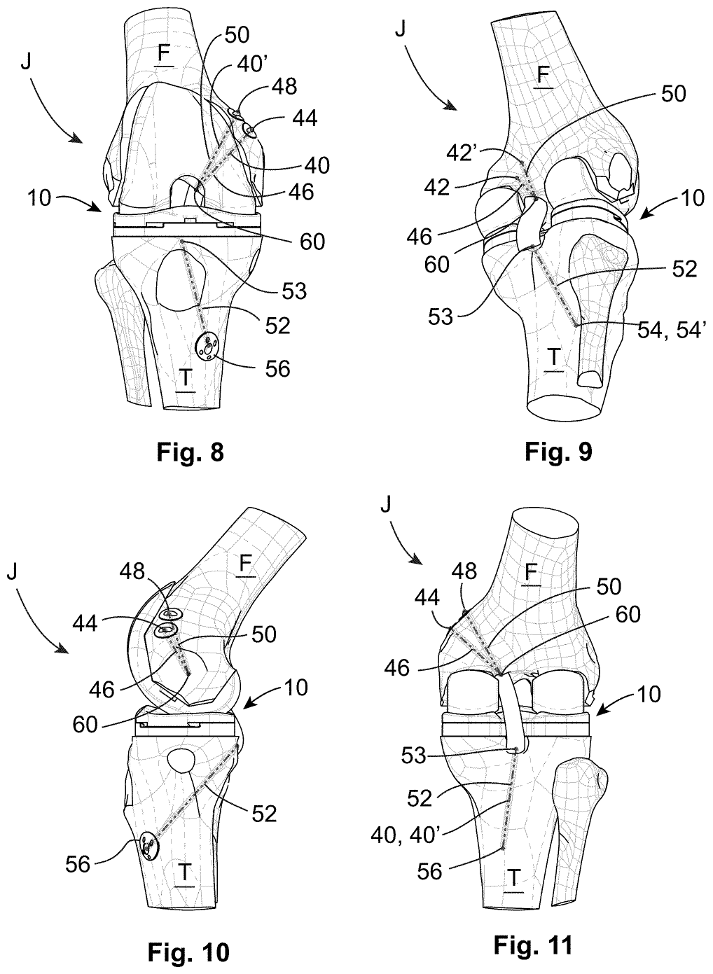

FIGS. 8-11 illustrate a tensile member passing through transosseous passaged formed in bone (e.g., by drilling), fixed by anchors, and routed across the posterior aspect of a human knee joint J. The tensile member replaces or augments or reinforces or tethers the PCL.

In the illustrated example, two tensile members are present, referred to as first and second tensile members 40, 40′ respectively.

The first tensile member 40 has a first end 42 secured to the femur F on the outboard side thereof, by a first anchor 44. (With reference to this example, the terms “inboard” and “outboard” are used to describe locations relative to their distance from the meeting articular surfaces of the joint J. For example, the implant 10 would be considered “inboard” of the joint J, while the anchor 44 would be considered “outboard”). The first tensile member 40 passes through a first femoral passage 46 formed in the femur F, exiting the inboard side of the femur F.

The second tensile member 40′ has a first end 42′ secured to the femur F on the outboard side thereof, by a second anchor 48. The second tensile member 40′ passes through a second femoral passage 50 formed in the femur F, exiting the inboard side of the femur F.

The first and second tensile members 40, 40′ span the gap between femur F and tibia T and enter a tibial passage 52 at an inboard side. The first and second tensile members 40, 40′ pass through the tibial passage 52 at a single entry 53, exiting the outboard side of the tibia T. Second ends 54, 54′ of the first and second tensile members 40, 42′ are secured with a third anchor 56.

The term “anchor” as it relates to elements 44, 48, and 56 refers to any device which is effective to secure a tensile member passing therethrough. Nonlimiting examples of anchors include washers, buttons, flip-anchors, adjustable loop devices, fixed loop devices, interference screw devices, screw plates, ferrules, swages, or crimp anchors. Examples of some particularly useful anchors are described below.

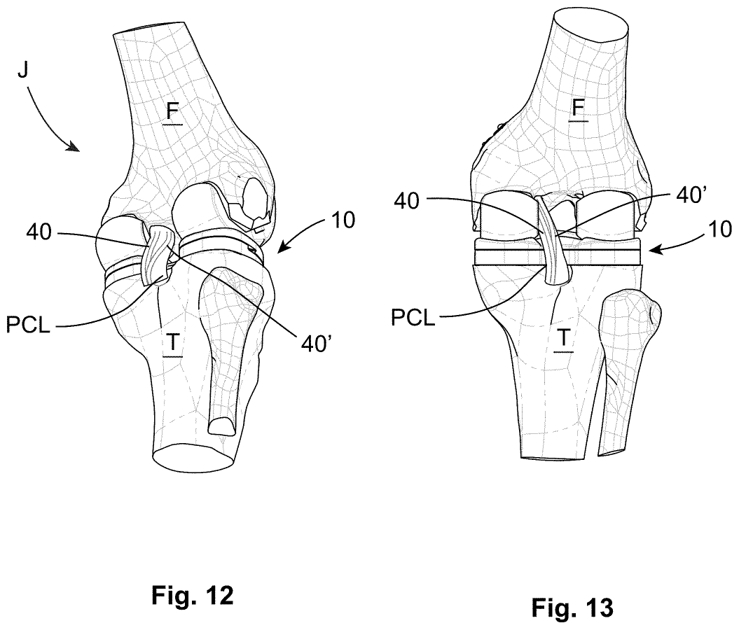

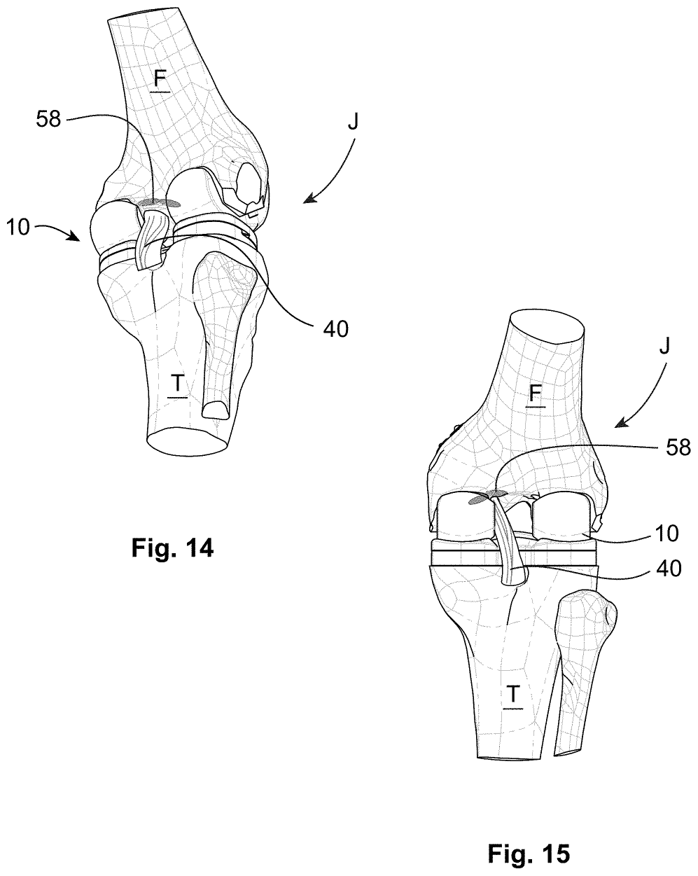

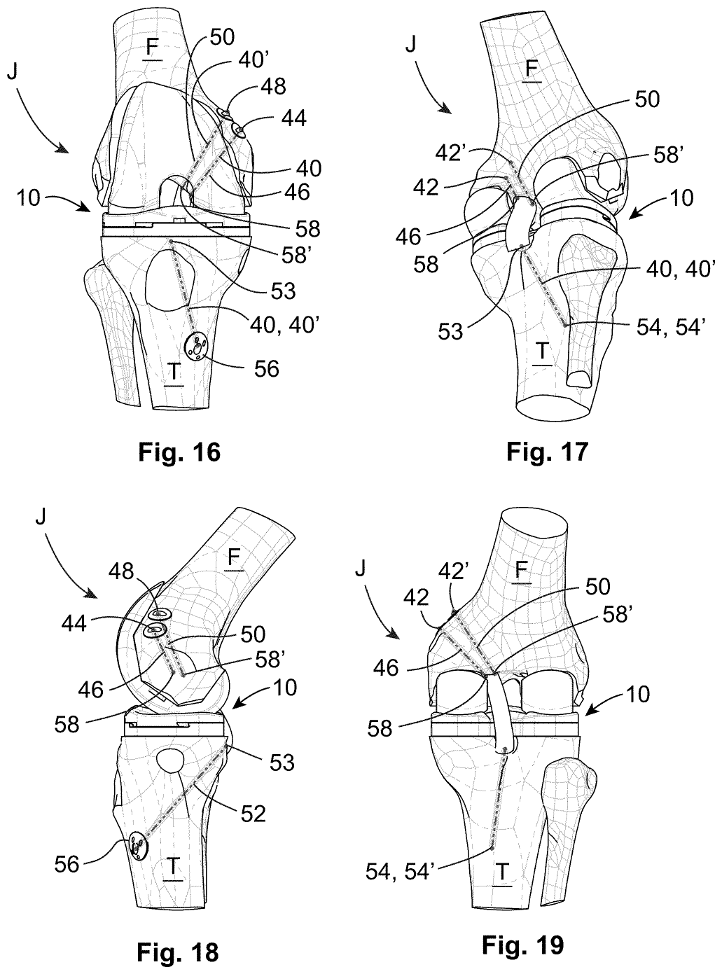

As seen in FIGS. 12 and 13, the tensile members 40, 40′ can be routed through or along the PCL. In one example, at least one of the ends of the first and second passages may be positioned inside a “footprint” defined by the portion of the cruciate ligament which contacts the femur. In another example, at least one end of the third passage may be positioned inside a “footprint” defined by the portion of the cruciate ligament which contacts the tibia. As seen in FIGS. 15 and 16, one or more of the anchors could be an internal anchor 58.

Analysis by the inventors has shown that the use of two or more tensile members 40 with non-coextensive transosseous routing can be especially helpful in providing a desired alignment and range of motion of the knee joint J. As used herein, the term “non-coextensive transosseous routing” refers to two or more tensile members passing through bone along different routes, i.e. two or more routes which are not coextensive over their entire lengths. Stated another way, the two or more transosseous passages would be either (a) intersecting at one location or (b) spaced-apart from the other transosseous passages and running along parallel or divergent paths.

In the example shown in FIGS. 8-11, the femoral passages 46, 50 intersect and have a single exit 60 at the articular surface of the medial condyle. In this example, both tensile members 40, 40′ pass through a single tibial passage 52. The tensile members 40, 40′ may have the same or different properties (e.g., material, diameter) and may have the same or different final implanted tensions.

In the example shown in FIGS. 16-19, the femoral passages 46, 50 do not intersect (they may be parallel or non-parallel). The femoral passages 46, 50 have individual exits 58, 58′ at the articular surface of the medial condyle. In this example, both tensile members 40, 40′ pass through a single tibial passage 52. The tensile members 40, 40′ may have the same or different properties (e.g., material, diameter) and may have the same or different final implanted tensions.

The exact routing of the femoral and tibial passages may be selected to suit a particular patient and desired correction. This may be determined using the surgeon's judgment, optionally supplemented by computer simulation of the knee joint. It will be understood that each tensile member 40, 40′ constrains the joint J in a predictable manner, and that the use of two tensile members 40, 40′ with non-coextensive routing produces a compound effect on the joint J.

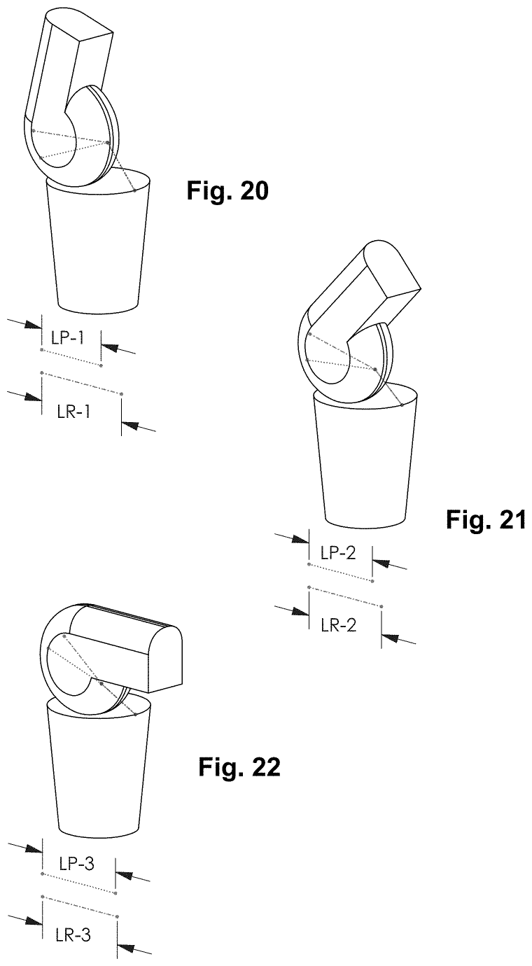

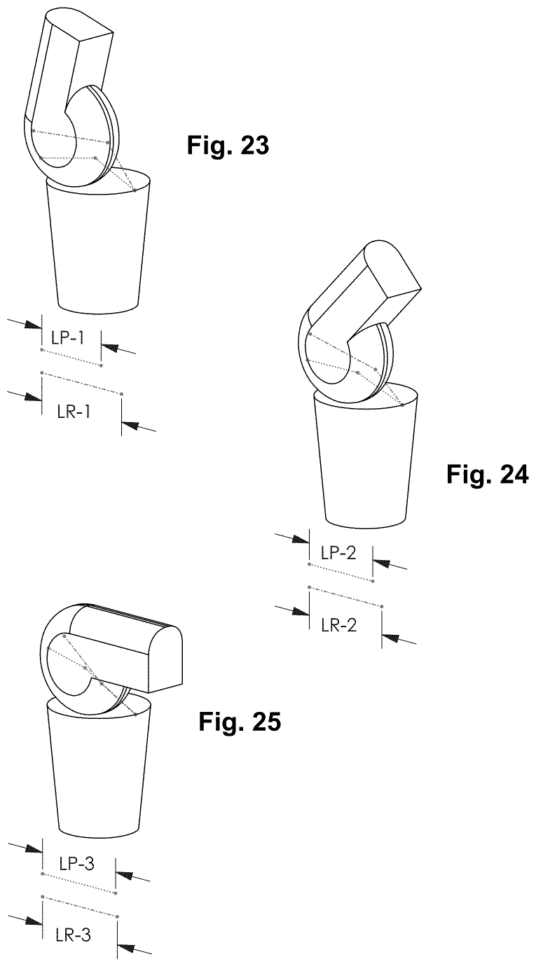

FIGS. 20-25 illustrate the effect of the non-coextensive routing. It can be seen that the changes in length of the elastic tensile members 40, 40′ as the joint J moves through extended, mid-flexed, and extended positions are different for the different routings. The dimension “LP” represents the overall length of one of the tensile members, and the dimension “LR” represents the overall length of the other tensile member. FIGS. 20-22 model routing similar to that shown in FIGS. 8-11, while FIGS. 23-25 model routing similar to that shown in FIGS. 16-19.

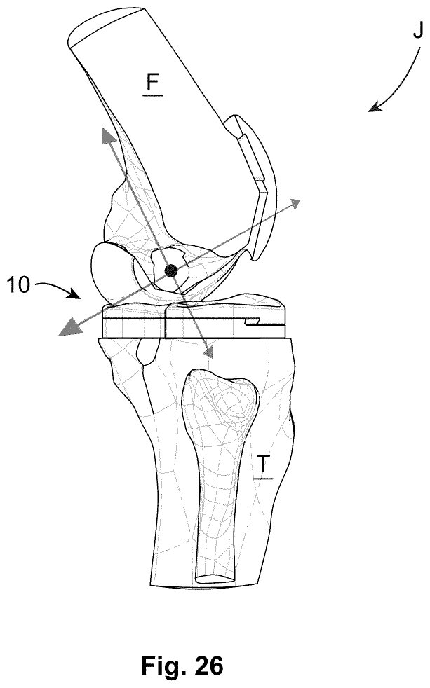

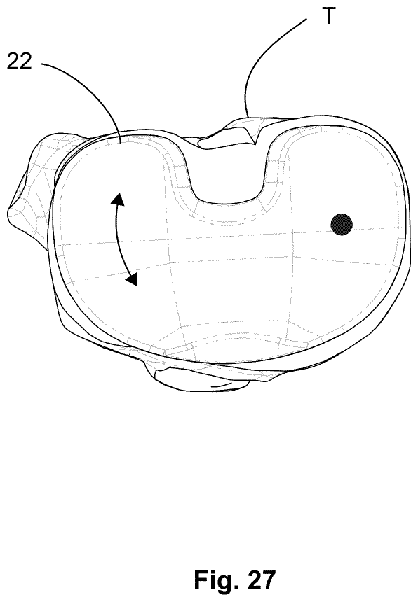

The cruciate retaining method described above can be employed for sagittal plane control. As shown in FIG. 26, appropriate selection, routing, and tensioning of the tensile members 40, 40′ can be used to move or constrain the femur F in different translational or rotational directions relative to the tibia, e.g., posterior, anterior, superior, or inferior translational directions; and internal or external rotational directions.

The cruciate retaining method described above can be employed for axial plane control. As shown in FIG. 27, appropriate selection, routing, and tensioning of the tensile members 10 can be used to move or constrain the femur F in different directions relative to the tibia, e.g., lateral, medial, posterior, or anterior translational directions; and internal or external rotational directions. Furthermore, appropriate selection, routing, and tensioning of the tensile members 10 can be used to control “medial pivot”, i.e., the magnitude to which the lateral condyle of the femur 10 moves and rotates in the anterior or posterior directions as the knee joint J is moved between flexed and extended positions.

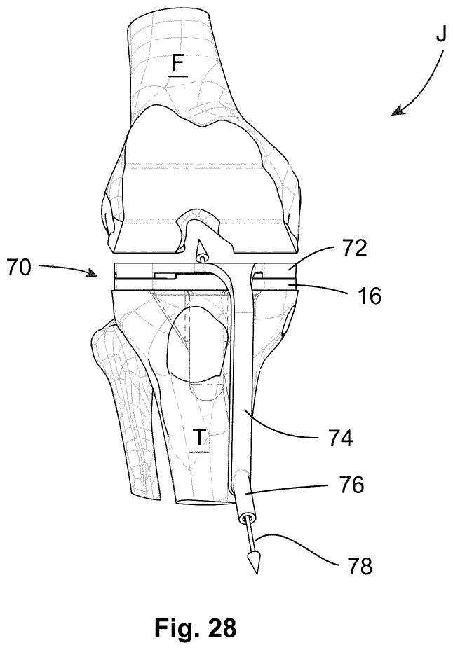

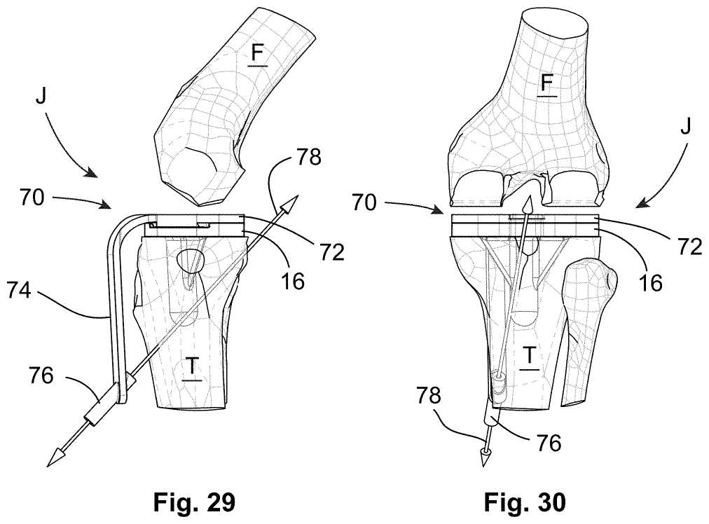

The tibial passage 52 may be oriented at a substantially acute angle to the surface of the tibia T and may thus be difficult to accurately drill using a manual process. FIGS. 28-30 illustrate a drill guide 70 which may be used to form the tibial passage 52. The drill guide 70 includes a base plate 72 which is shaped and sized to engage with a tibial tray 16. A support arm 74 extends away from the base plate 72 in the inferior direction anatomically. A drill bushing 76 is disposed at the far end of the support arm 74. The drill bushing 76 has an internal bore for receiving a drill bit or other similar boring tool. The drill bushing 76 is positioned and oriented so as to guide the boring tool along a path to create the tibial passage 52, as shown by the line 78 in the figures.

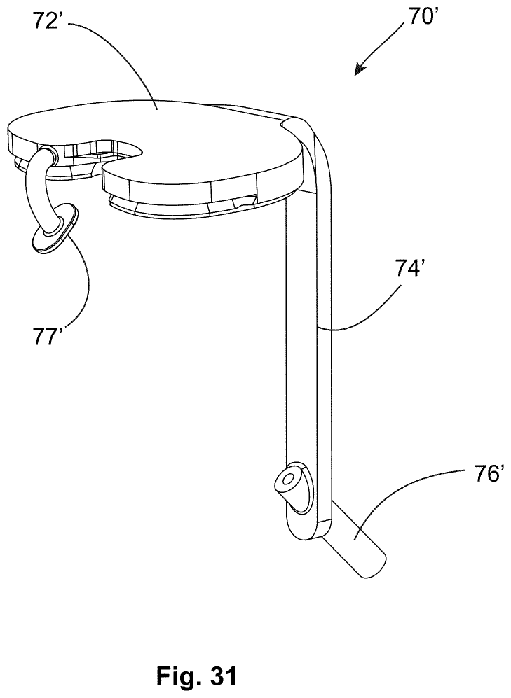

FIG. 31 illustrates a modified version of the drill guide, labeled 70′, including a base plate 72′, support arm 74′, and drill busing 76′. The drill guide 70′ includes a drill stop 77′ extending from the base plate 72′. It is positioned and oriented in line with the bore axis of the drill bushing 76′. The drill stop 77′ is effective to stop the boring tool after it has passed through the tibia T, preventing damage to other anatomical structures.

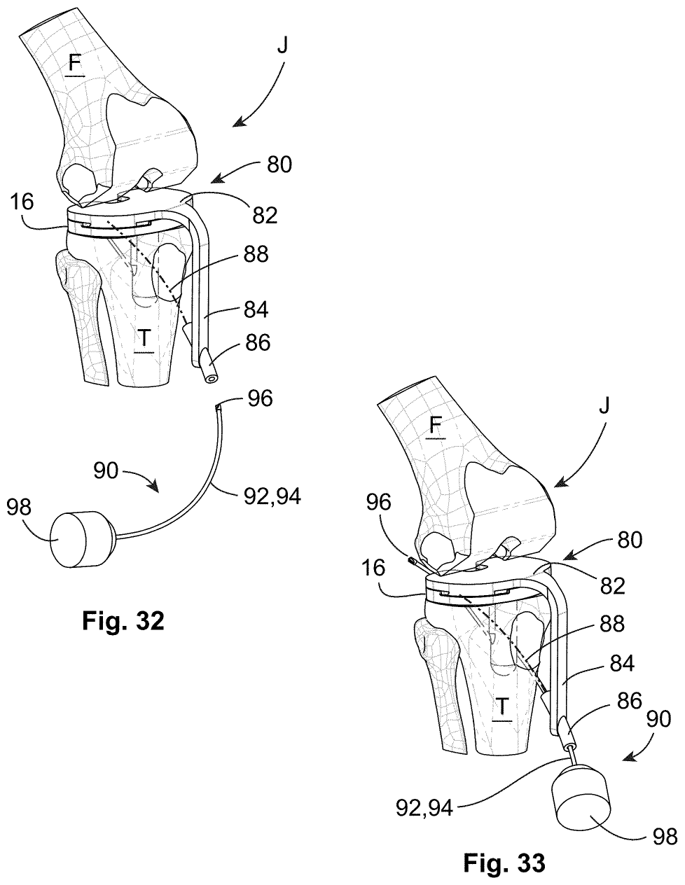

FIGS. 32 and 33 illustrate another drill guide 80 which may be used to form the tibial passage 52. The drill guide 80 includes a base plate 82 which is shaped and sized to engage with a tibial tray 16. A support arm 84 extends away from the base plate 82 in the inferior direction anatomically. A drill bushing 86 is disposed at the far end of the support arm 84. The drill bushing 86 has an internal bore for receiving a drill bit or other similar boring tool. The drill bushing 86 is positioned and oriented so as to guide the boring tool along a path to create the tibial passage 52 as shown by the line 88 in the figures. The drill bushing 86 has a curved bore, in contrast to the straight axial bore of the drill bushing 76 shown in FIGS. 28-30.

The drill guide 80 is used in conjunction with a flexible drill 90. The flexible drill 90 has a generally stiff non-rotating central support member 92 surrounded by a flexible member 94 (similar to a coil spring). The flexible member 94 terminates in a cutting tip 96 and is driven by a drill motor 98 (shown schematically).

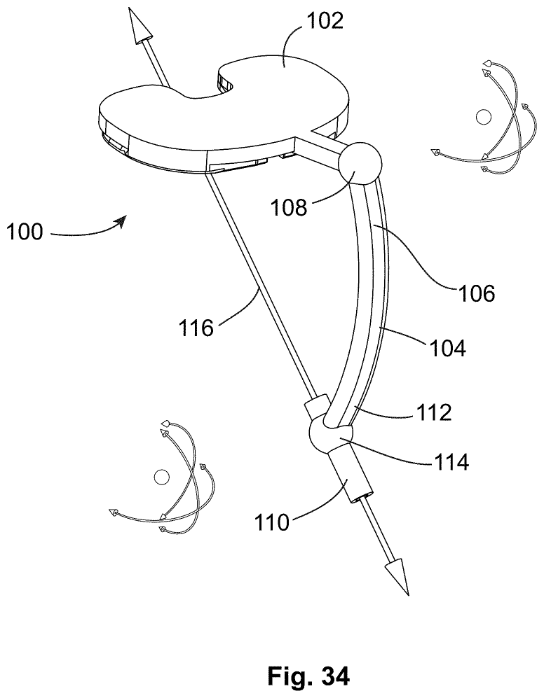

FIG. 34 illustrates an adjustable drill guide 100 which may be used to form the tibial passage 52. The drill guide 100 includes a base plate 102 which is shaped and sized to engage with a tibial tray 16. A support arm 104 extends away from the base plate 102 in the inferior direction anatomically. A first end 106 of the support arm 104 is connected to the base plate 102 with a first pivoting joint 108. A drill bushing 110 is disposed at the far end 112 of the support arm 102, and is connected to the far end 112 of the support arm 102 by a second pivoting joint 114. The pivoting joints 108, 114 are configured to permit the connected elements to be relatively pivoted about one or more axes, and to retain the connected parts in the pivoted positions. For example, they may incorporate a clamp, lock, or friction mechanism (not shown in detail). The drill bushing 110 has an internal bore for receiving a drill bit or other similar boring tool. The drill bushing 110 is positioned and oriented so as to guide the boring tool along a path to create the tibial passage, as shown by the line 116 in the figures.

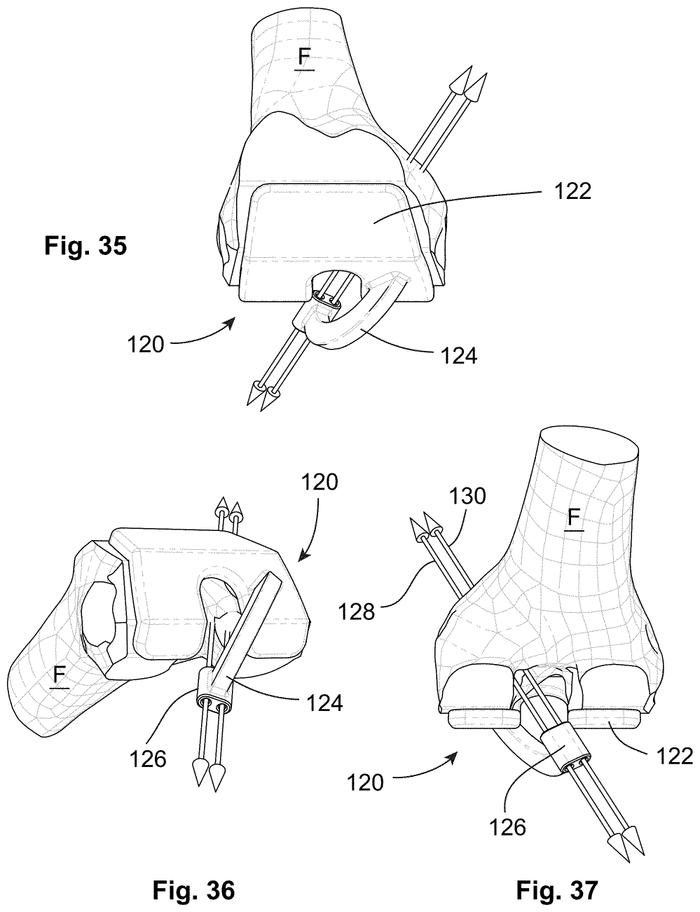

FIGS. 35-37 illustrate another drill guide 120 which may be used to form the femoral passages 46, 50. The drill guide 120 includes a base plate 122 which is shaped and sized to engage the distal end of the femur F. For example, it may be shaped complementary to the shape of a femur F that has been prepared for an implant. A support arm 124 extends away from the base plate 122 in the superior direction anatomically. A drill bushing 126 is disposed at the far end of the support arm 124. The drill bushing 126 has one or more internal bores for receiving a drill bit or other similar boring tool. The drill bushing 126 is positioned and oriented so as to guide the boring tool along a path to create the femoral passages 46, 50 as shown by the lines 128, 130 in the figures.

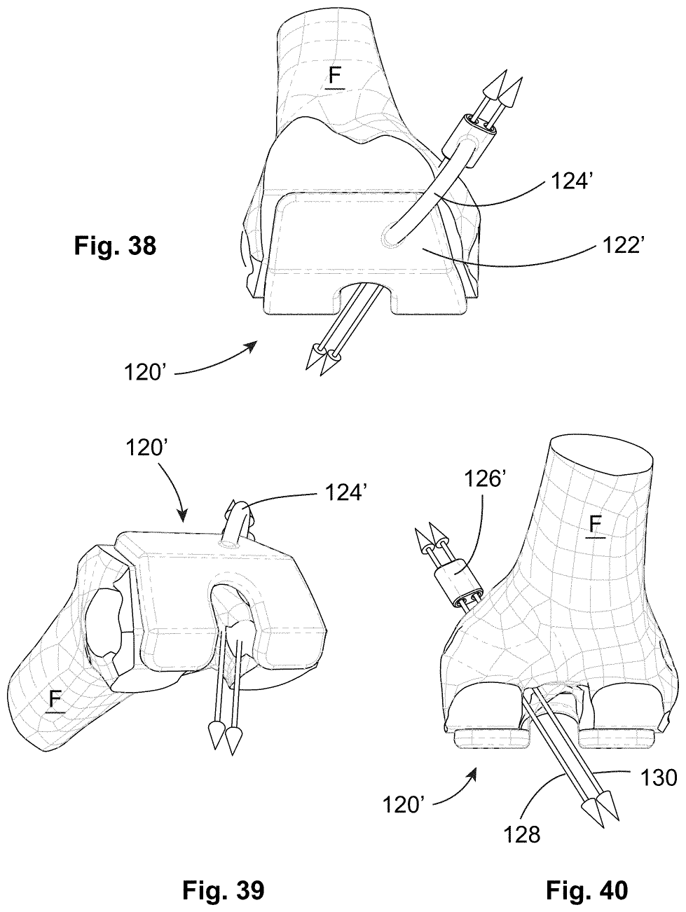

The drill guide 120 is intended for a approach from the inferior direction. FIGS. 38-40 illustrate a drill guide 120′ which may be used to form the femoral passages 46, 50 in an approach from the superior direction. The drill guide 120′ includes a base plate 122′, support arm 124′, and a drill bushing 126′. The drill bushing 126 is positioned and oriented so as to guide the boring tool along a path to create the femoral passages 46, 50 as shown by the lines 128, 130. in contrast to drill guide 120, the support arm 124′ of drill guide 120′ extends away from the base plate 122′ in the opposite direction.

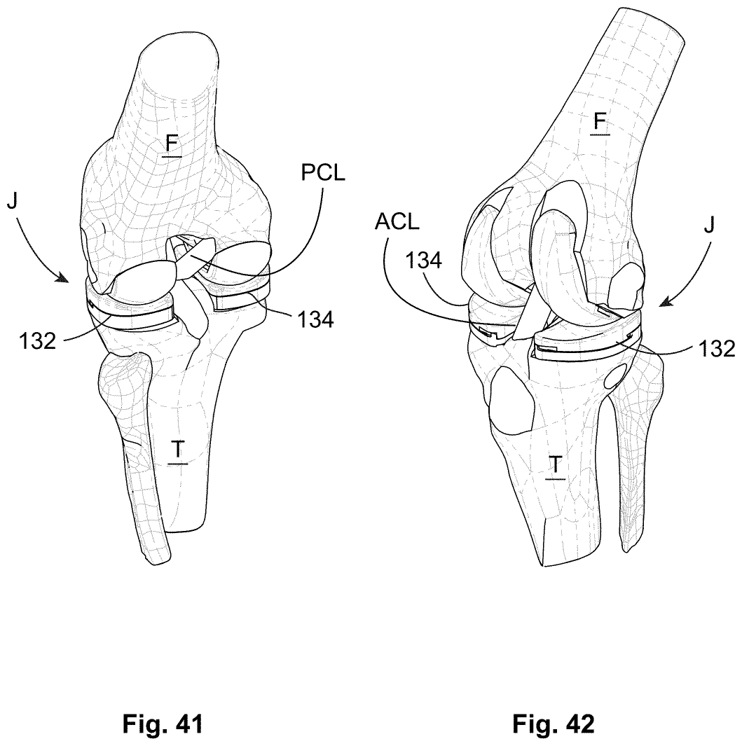

The cruciate-preserving and augmenting and reinforcing techniques described herein are applicable to varying scopes of knee surgery. It may be used in combination with resurfacing of all or part of the knee with artificial or biological materials. It may be used in combination with a knee arthroplasty. The endoprosthesis used in the arthroplasty may be a total knee, using the implant 10 of FIGS. 5 and 6, or it may be a bicompartmental arthroplasty, using individual implants 132, 134 for the medial and lateral compartments, as seen in FIGS. 41 and 42. Alternatively, the arthroplasty may be uni-compartmental. As a further alternative, the cruciate-preserving and augmenting techniques described herein are useful even where resurfacing is not carried out, for example other surgical procedures to restore the function of the knee joint.

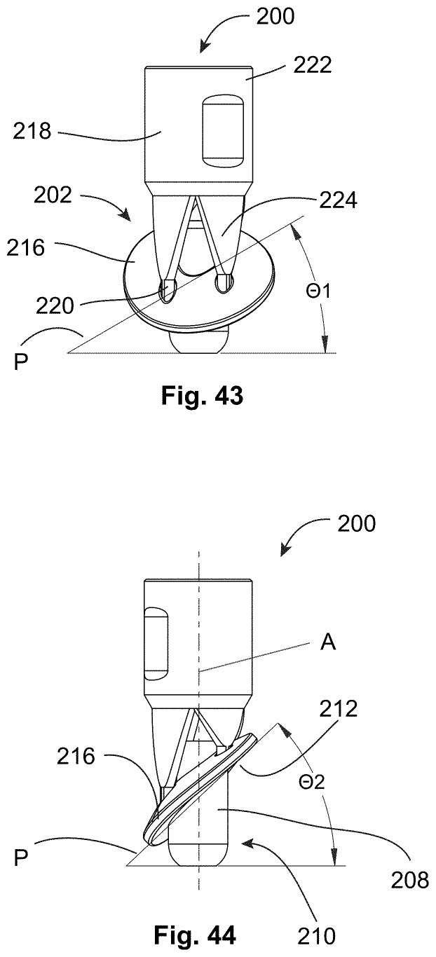

FIGS. 43-47 illustrate an exemplary embodiment of an anchor 200. The anchor 200 includes three functional elements, namely a housing 202 configured to be implanted into bone, a collet 204 received in the housing 202 and configured to be swaged around and against a tensile member 40 (FIG. 7) without moving axially relative to the housing 202 or tensile member 40, and a sleeve 206 received in the housing 202 which is capable of moving axially within the housing 202 so as to swage the collet 204, thus retaining the tensile member 40. (Some minimal axial movement of the collet 204 not significantly affecting tension may occur during swaging).

The housing 202 has a body portion 208 extending along a central axis “A” between first and second ends 210, 212. The body portion 208 is defined by a peripheral wall having opposed interior and exterior surfaces, and defining a hollow interior. In the illustrated example, the body portion 208 is generally cylindrical in shape. The first end 210 has an internal flange 214 which is sized to define a stop against axial motion of the collet 204.

A generally annular flange 216 is located at or near the second end 212 and extends radially outwards from the body portion 208. The size and shape of the flange 216 may be selected to suit a particular application. In the example illustrated in FIGS. 43 and 44, a reference plane “P” passing through the flange 216 is oriented at a compound angle oblique to the central axis A, represented by angles θ1, 02 in two perpendicular planes. It will be understood that the orientation of the flange 216 may be varied to suit a particular application. The anchor 200 may have an overall size which is generally small enough to be implanted inside a human body. In one example the housing 202 may be cylindrical in shape with an outside diameter “D2” of about 3 to 12 mm, and the flange 216 may have an outside diameter “D3” about 5 to 20 mm.

The housing 202 includes an extension portion 218 extending away from the second end 212 of the body portion 208. The extension portion 218 is coupled to the body portion 208 by a breakaway structure 220. As manufactured and prior to use, the entire housing 202 forms a single unitary, integral, or monolithic structure including the body portion 208, extension portion 218, and breakaway structure 220 that provides a “breakaway” or “snap-off” connection between the body portion 208 and the extension portion 218.

The extension portion 218 extends between a first end 222 and a second end 224. The second end 224 is interconnected to the breakaway structure 220. The first end 222 may be provided with a mechanical connector for being connected to an insertion instrument which is described in more detail below. In the illustrated example, the first end 222 is provided with a connector 226 in the form of screw threads. As described in more detail below, this permits a secure, releasable connection to an instrument used for insertion or manipulation of the anchor 200.

The breakaway structure 220 is configured in terms of its shape, dimensions, and material properties such that it will retain its structural integrity and interconnected the body portion 208 and the extension portion 218 when subjected to tensile loads up to a first magnitude sufficient to complete a swaging process of the anchor 200 as described below. This is referred to herein as a “first predetermined tensile load”. The breakaway structure 220 is further configured in terms of its shape, dimensions, and material properties such that it will fail and permit separation of the body portion 208 and the extension portion 218 when subjected to tensile loads equal to or greater than a second magnitude, referred to herein as a “second predetermined tensile load”. The second tensile load is greater than the first tensile load. The second tensile load may be selected to be sufficiently greater than the first predetermined tensile load such that failure of the breakaway structure 220 is unlikely to occur during the swaging process. Stated another way, the second predetermined tensile load may have a safety margin over the first predetermined tensile load. In one example, the second predetermined tensile load may be selected to be at least 50% to 100% greater than the first predetermined tensile load.

In general, the breakaway structure 220 may include one or more stress-concentrating columns which present a known cross-sectional area, thus permitting reliable computation of the tensile stresses in the breakaway structure 220 for a given applied load.

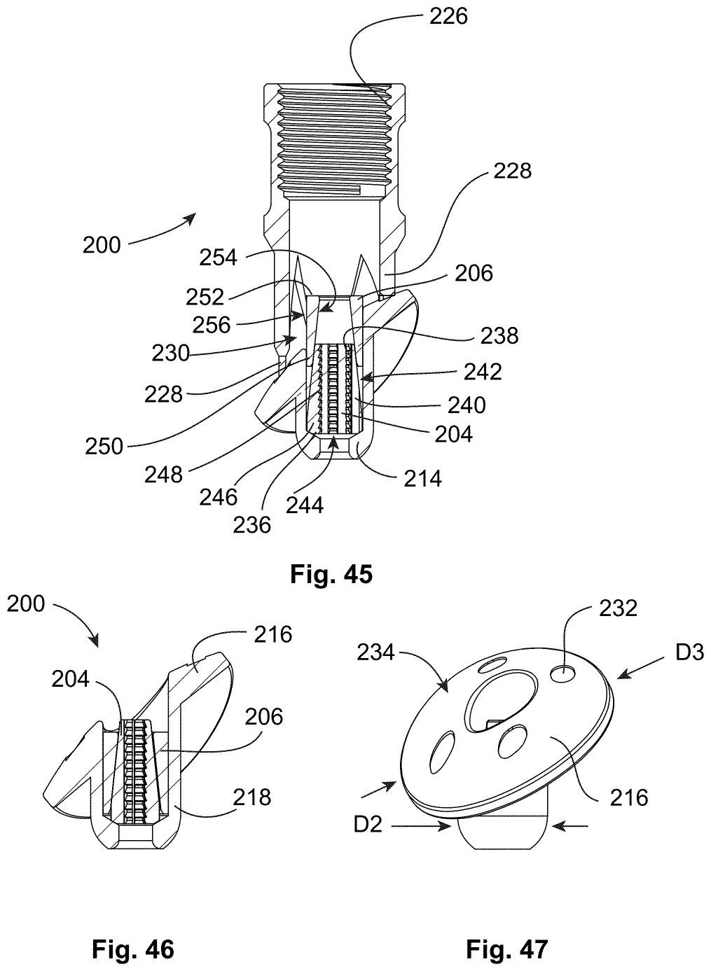

In the illustrated example, best seen in FIG. 45, the breakaway structure 220 includes a plurality of stress-concentrating columns 228 arrayed around the periphery of the flange 216, which have a circular cross-sectional shape adjacent to and/or or at the flange 216. The stress-concentrating columns 228 are separated by openings 230. It will be understood that other column cross-sectional shapes providing a predictable cross-sectional area may be used, and that the cross-sectional shape may vary over the length of the column.

Optionally, the stress-concentrating columns 228 may intersect the flange 216 at the bottom of recesses 232 formed in the flange 216. In use, this permits the stress-concentrating columns 228 to separate along the fracture plane which is “below” a top surface 234 of the flange 216 or stated another way it is sub-flush to, or recessed from, the top surface 234.

The collet 204 is a hollow member with first and second ends 236, 238 and defined by a sidewall 240 having an exterior surface 242. The collet 204 has a central bore 244 which is sized to receive the tensile member 40 described above. For example, the central bore 244 may be cylindrical, with a minimum inside diameter or characteristic dimension which is initially slightly larger than a diameter D1 of the tensile member 40. The central bore 244 need not have a circular cross-section; the cross-section may be a polygon shape (e.g. triangular, square) or it may be a lobed shape (e.g., triangular with radiused corners).

The collet 204 is configured so as to readily permit it to be swaged, i.e. shaped in such a manner to reduce its cross-section and the size of the central bore 244 so that it firmly engages the tensile member 40 and allows a tensile force to be applied thereto. The act of swaging may involve the collet 204 being deformed, crushed, collapsed, or compressed. The collet 204 is configured, e.g., sized and shaped, such that when subjected to pressure from the sleeve 206, it will abut the internal flange 214 of the body portion 208, thus stopping its further axial movement, and permitting the swaging action to take place without axial movement of the collet 204 relative to the tensile member 40 or housing 202.

The exterior surface 242 has a shape adapted to interact with the interior surface of the sleeve 206 described below so as to produce a radially inwardly directed force on the collet 204 in response to the axial movement of the sleeve 206. Fundamentally, at least one of the exterior surface 242 of the collet 204 and the interior surface of the sleeve 206 incorporates a taper i.e., a diameter or lateral dimension which is larger near one end and smaller near the opposite end of the respective element. In the example shown in FIG. 45, the exterior surface 242 has a cylindrical section 246 and a generally frustoconical section 248. The exterior dimensions and shape of the exterior surface 242 are selected so as to provide a predetermined fit with the sleeve 206 both before and after a compression process. FIG. 61 shows the collet 204 after swaging.

The central bore 244 may include a surface texture which serves to enhance grip on a tensile member 40. Nonlimiting examples of surface texture structures include teeth, ribs, grooves, dimples, recesses, bumps, pins, ridges, knurling, checkering, and threads. In the example shown in FIG. 45 this takes the form of longitudinal rows of ramped-shaped teeth.

The sleeve 206 is a hollow member with open first and second ends 250, 252. The sleeve 206 is sized is such that the tensile member 40 described above can pass through the first and second ends 250, 252. The sleeve 206 is defined by a peripheral wall having interior and exterior surfaces 254, 256, respectively. In the illustrated example, the sleeve 206 is generally cylindrical in shape.

The interior surface 254 has a shape adapted to interact with the exterior surface 242 of the collet 204 described above so as to produce a radially inwardly directed force on the collet 204 in response to the axial movement of the sleeve 206. As noted above, at least one of the exterior surface 242 of the collet 204 and the interior surface 254 of the sleeve 206 incorporates a taper i.e., a diameter or lateral dimension which is larger near the first end and smaller near the second end of the respective element. In the example shown, the interior surface 254 is tapered, defining a shape like a frustum of a cone, with a larger diameter at the first end 250.

The anchor 200 and its components may be made from any material which is biocompatible and which will engage the other elements so as to transfer tensile force thereto. As used herein, the term “biocompatible” refers to a material which is not harmful to living tissue. Nonlimiting examples of suitable materials include polymers and metal alloys. Nonlimiting example of suitable metal alloys include stainless steel alloys and titanium alloys. The anchor 200 or its components may be fabricated by a technique such as machining, forging, casting, sintering, or additive manufacturing (e.g., “3D printing”). The anchor 200 or its components may be treated with known coating such as titanium nitride, chrome plating, carbon thin films, and/or diamond-like carbon coatings.

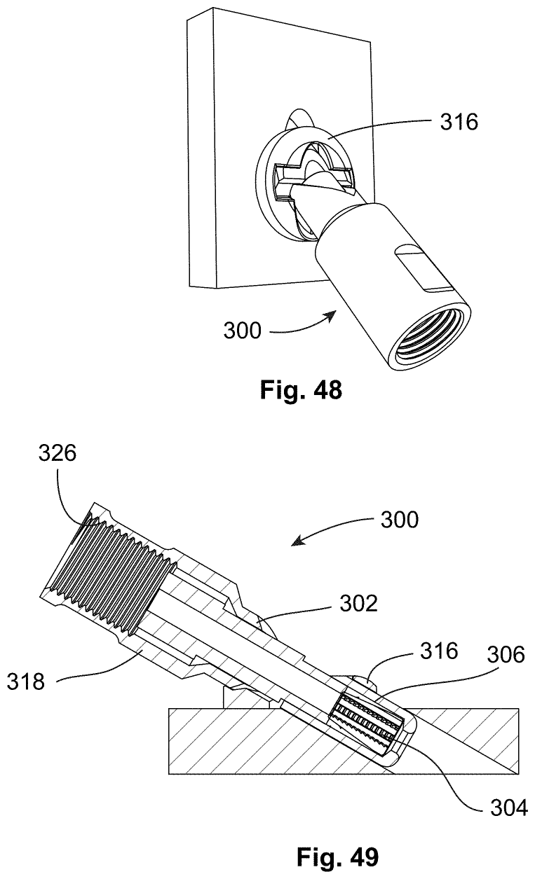

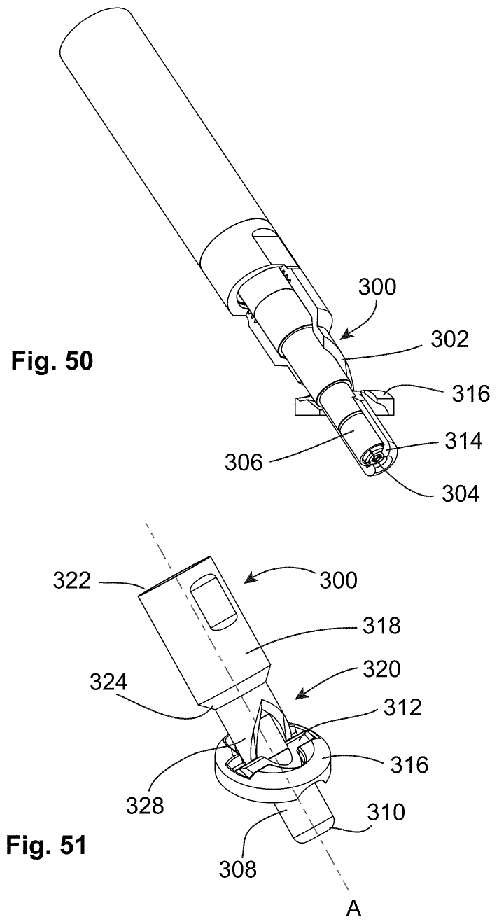

FIGS. 48-54 illustrate an exemplary embodiment of an anchor 300 which incorporates a pivoting function. The anchor 300 is similar in overall construction to the anchor 300 described above. Elements of the anchor 300 not specifically described may be taken to be identical to corresponding elements of the anchor 200. The anchor 300 includes a washer 316, a housing 302, a collet 304 received in the housing 302 and configured to be swaged around and against a tensile member, and a sleeve 306 received in the housing 302 which is capable of moving axially within the housing 302 so as to swage the collet 304, thus retaining the tensile member 40.

The housing 302 has a body portion 308 extending along a central axis “A” between first and second ends 310, 312. The body portion 308 is defined by a peripheral wall having opposed interior and exterior surfaces, and defining a hollow interior. In the illustrated example, the body portion 308 is generally cylindrical in shape. The first end 310 has an internal flange 314 which is sized to define a stop against axial motion of the collet 304.

A pair of trunnions 315 extend laterally outwards from the body portion 308 at or near the second end 312. The trunnions 315 may include surfaces which are wholly or partially arcuate or cylindrical.

The housing 302 includes an extension portion 318 extending away from the second end 312 of the body portion 308. The extension portion 318 is coupled to the body portion 308 by a breakaway structure 320. In the illustrated example, the breakaway structure 320 includes stress-concentrating columns 328 which join to the trunnions.

The extension portion 318 extends between a first end 322 and a second end 324. The second end 324 is interconnected to the breakaway structure 320. The first end 322 may be provided with a mechanical connector 326 for being connected to an insertion instrument, such as screw threads.

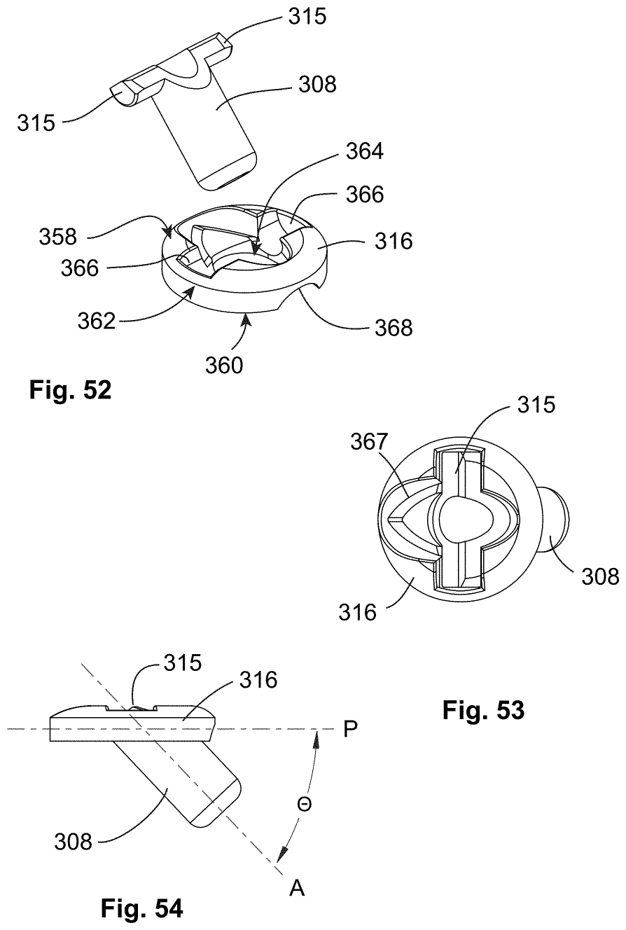

The washer 316 is shaped like a disk and has an upper side 358, an opposed lower side 360, and a peripheral surface 362 interconnecting the upper and lower sides 358, 360. The washer 316 may be considered a “flange element”. An aperture 364 shaped and sized to receive the body portion 308 passes through the washer 316. The washer 316 includes a pair of spaced-apart pivot recesses 366 which are complementary to the trunnions 315. When the body portion 308 is assembled into the washer 316, it can pivot about the trunnions 315. Depending on how the body portion 308 is pivoted, a reference plane “P” passing through the washer 316 may be oriented perpendicular to the central axis A, or at an angle θ oblique to the central axis A. This pivoting action accommodates a range of hole angles relative the bone cortical surface. As best seen in FIG. 52, the upper and lower sides 358, 360 may incorporate reliefs 367, 368 to permit pivoting of the body portion 308 to angles at the extreme ends of its range.

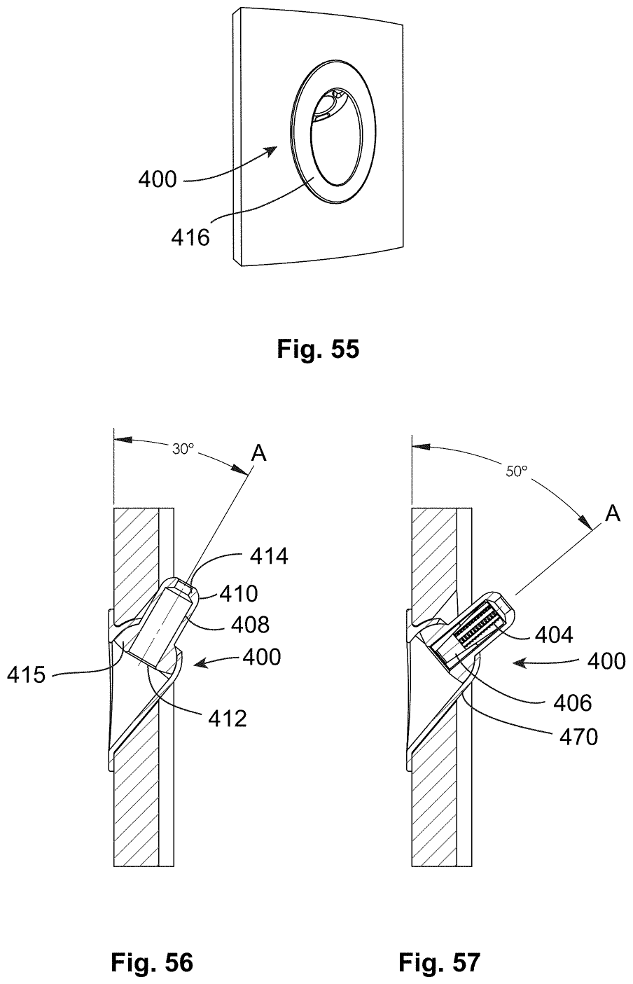

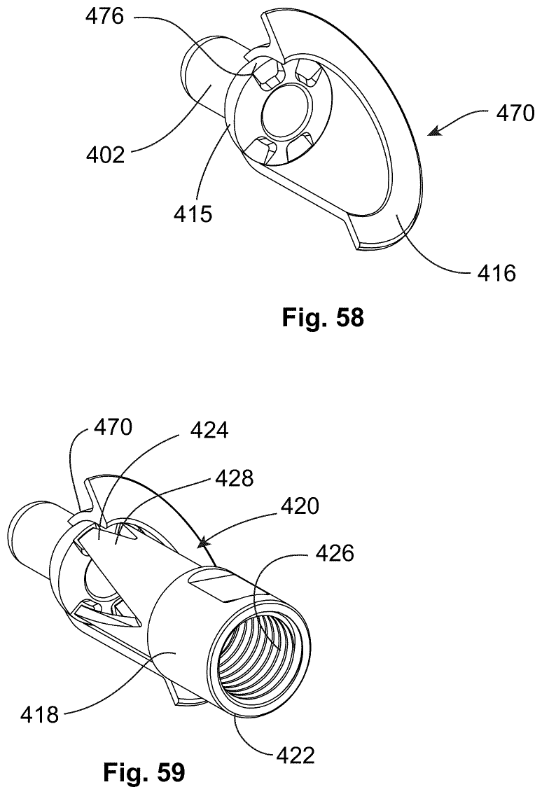

FIGS. 55-59 illustrate an exemplary embodiment of an anchor 400 which incorporates a pivoting function. The anchor 400 is similar in overall construction to the anchor 400 described above. Elements of the anchor 400 not specifically described may be taken to be identical to corresponding elements of the anchor 200. The anchor 400 includes a housing 402, a collet 404 received in the housing 402 and configured to be swaged around and against a tensile member, and a sleeve 406 received in the housing 402 which is capable of moving axially within the housing 402 so as to swage the collet 404, thus retaining the tensile member 40.

The housing 402 has a body portion 408 extending along a central axis “A” between first and second ends 410, 412. The body portion 408 is defined by a peripheral wall having opposed interior and exterior surfaces, and defining a hollow interior. In the illustrated example, the body portion 408 is generally cylindrical in shape. The first end 410 has an internal flange 414 which is sized to define a stop against axial motion of the collet 404.

An annular ball surface 415 extends laterally outwards from the body portion 408 at or near the second end 412.

The housing 402 includes an extension portion 418 extending away from the second end 412 of the body portion 408. The extension portion 418 is coupled to the body portion 408 by a breakaway structure 420. In the illustrated example, the breakaway structure 420 includes stress-concentrating columns 428 which join to the trunnions.

The extension portion 418 extends between a first end 422 and a second end 424. The second end 424 is interconnected to the breakaway structure 420. The first end 422 may be provided with a mechanical connector 426 for being connected to an insertion instrument, such as screw threads.

The housing 402 is received in a hollow cup 470 having first and second ends 472, 474. The first end 472 of the cup 470 defines a concave seat 476 which is complementary to the ball surface 415 of the body portion 408. Thus assembled, the housing 402 can pivot to various orientations relative to the cup, as seen in FIGS. 56 and 57. A generally annular flange 416 is located at or near the second end 474 of the cup 470. The cup 470 may be considered a “flange element”.

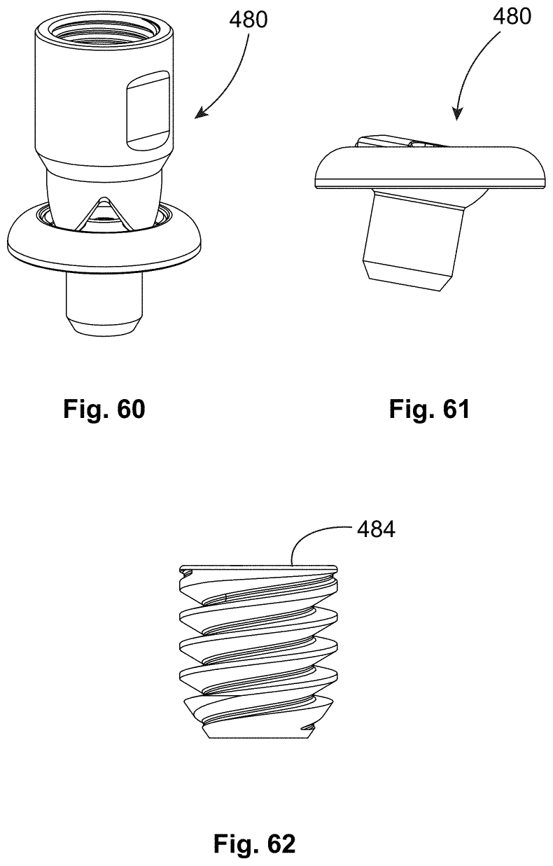

FIGS. 60-62 illustrate some possible alternative anchors for the tensile member 40. FIGS. 60 and 61 illustrate an anchor 480 with a ball pivot structure similar to anchor 400, but lacking the deep sub-cortical cup. FIG. 62 illustrates an anchor 484 with an externally-threaded housing.

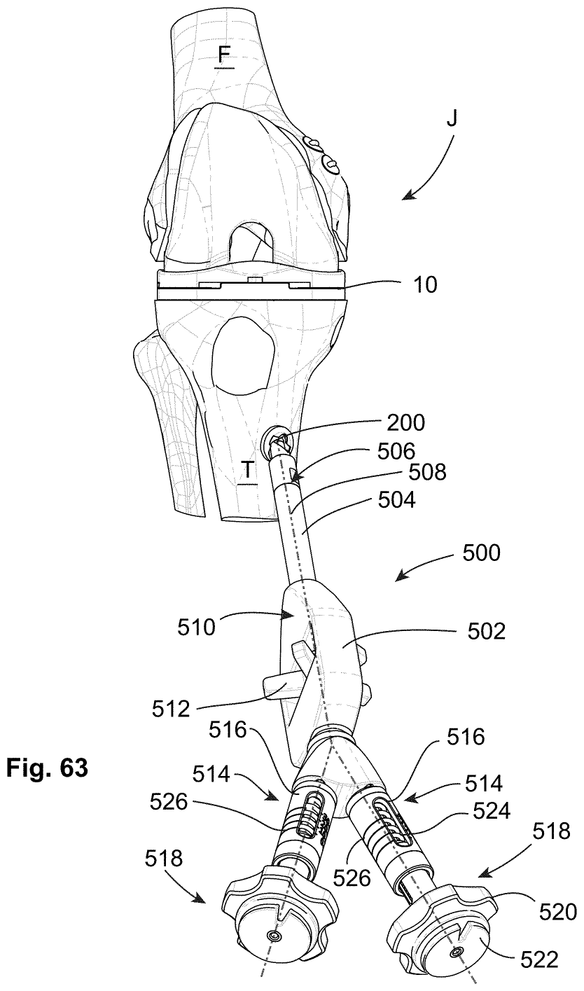

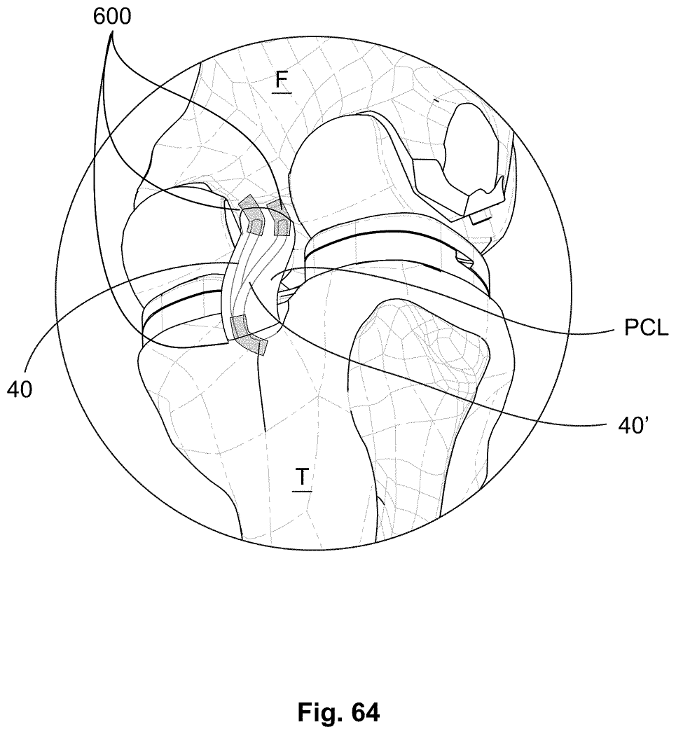

FIG. 63 illustrates an exemplary insertion instrument 500 which may be used to insert, tension, and activate any of the swage-type anchors described above. The basic components of the insertion instrument 500 are a body 502, a stem 504 extending from the body 502 and having an anchor connection mechanism 506 disposed at a distal end thereof, a hollow pushrod 508 extending through the stem 504 and slidably movable between retracted and extended positions, and a driving mechanism 510 for moving the pushrod 508 between retracted and extended positions. The stem 504 and the pushrod 508 may be rigid or flexible.

In the illustrated example, the driving mechanism 510 comprises an internal threaded mechanism which is manually operated by a star wheel 512.

A tensioner 514 is part of or connected to the insertion instrument 500. It has a housing 516. A shuttle assembly 518 including an adjustment knob 520 and a grooved spool 522 is received inside the housing 516. A compression spring 524 is captured between the shuttle assembly 518 and the housing 516. The shuttle assembly 518 can translate forward and aft relative to the housing 516 in response to rotation of the adjustment knob 520.

In use, a first end of a tensile member 40 passes through the hollow interior of tensioner 514 and is secured to the spool 522. The tension applied to the tensile member 40 may be indicated, for example, by observing the position of the shuttle assembly 518 relative to a calibrated scale 526 on the housing 516. When a suitable final tension is achieved, the star wheel 512 may be operated to actuate the pushrod 508, swaging the tensile member 40 and fracturing the breakaway structure of the anchor. In the illustrated example, two separate tensioners 514 are provided, allowing the tension of each of the tensile members to be set independently.

In one example procedure where two tensile members are used, a first provisional tension is applied to the first tensile member and a second provisional tension is applied to the second tensile member. The second tensile member may have the same or different tension at the first tensile member. Next, the provisional tensions evaluated to determined if they are suitable. In response to the evaluation they may be increased or decreased. Finally, the anchor may be swaged to secure the tensile members and finalize the tension. In one example, the tension may be from about 0 kg (222 N) to about (50 lb.)

FIG. 64 illustrates protective sleeves 600 which may be implanted at the exit holes on the inboard sides of the passages through the femur F and/or tibia T. The sleeves 600 are wear-resistant semi-flexible members and may be constructed from a material such as a biocompatible or bio-resorbable polymer. Tensile members 40, 40′ are routed through the sleeves. In use, they extend “around the bend” of the exit hole to prevent degenerative action of the tensile member 40 against the exit hole in the bone.

The apparatus and method described herein have numerous advantages over prior art apparatus and techniques. They provide a desirable knee outcome, give the ability to have a reinforced PCL with uni-condylar constructs, and have a potential for long term cost reduction, compared to prior art cruciate-sacrificing methods. Additionally, a PCL-preserving or reinforcing knee arthroplasty technique may contribute to better patient outcomes, higher patient satisfaction, and a reduction in the cost of overall patient health.

The foregoing has described apparatus and methods for reinforced knee arthroplasty. All of the features disclosed in this specification, and/or all of the steps of any method or process so disclosed, may be combined in any combination, except combinations where at least some of such features and/or steps are mutually exclusive.

Each feature disclosed in this specification may be replaced by alternative features serving the same, equivalent or similar purpose, unless expressly stated otherwise. Thus, unless expressly stated otherwise, each feature disclosed is one example only of a generic series of equivalent or similar features.

The invention is not restricted to the details of the foregoing embodiment(s). The invention extends, or to any novel one, or any novel combination, of the steps of any method or process so disclosed.

What is claimed is:

1. An anchor for anchoring one or more tensile members to bone, comprising:
a housing extending along a central axis between open first and second ends, and having a hollow interior;
a collet disposed in the hollow interior of the housing, the collet having a peripheral wall defining a central bore for accepting one or more tensile members therethrough and an exterior surface, wherein the collet is configured to be swaged around and against the one or more tensile members;
a sleeve having a peripheral wall defining opposed interior and exterior surfaces, the sleeve disposed in the hollow interior of the housing and positioned generally axially adjacent to the collet, so as to be movable parallel to the central axis between first and second positions;
wherein at least one of the exterior surface of the collet and the interior surface of the sleeve is tapered and the sleeve and the collet are arranged such that movement of the sleeve from the first position to the second position will cause the interior surface of the sleeve to bear against the exterior surface of the collet, causing the collet to swage radially inwards around and against the one or more tensile members; and
a flange element, wherein the housing is pivotally mounted to the flange element.

2. The anchor of claim 1 wherein the housing includes a body portion and an extension portion interconnected by a breakaway connection which is configured to retain structural integrity under a first predetermined tensile load and to separate under a second predetermined tensile load which is greater than the first predetermined tensile load.

3. The anchor of claim 1 further comprising a surface texture configured to enhance grip on a tensile member formed in the central bore.

4. The anchor of claim 1, in combination with an insertion instrument comprising:
a body configured to be connected to the housing;
a hollow pushrod extending through the stem and slidably movable between retracted and extended positions;
a driving mechanism operable to move the pushrod between the retracted and extended positions;

5. The combination of claim 4, wherein the insertion instrument further comprises: a tensioner including a housing coupled to the body of the insertion instrument, the housing including a shuttle which is configured to engage a tensile member and which is selectively movable relative to the housing.

6. The anchor of claim 1, wherein:
the flange element is a washer; and
The housing includes laterally-extending trunnions which are received in pivot recesses of the washer, such that the housing can pivot about the trunnions.

7. The anchor of claim 6, wherein the trunnions include surfaces which are at least partially cylindrical.

8. The anchor of claim 6, wherein the housing includes:
a body portion extending between first and second ends; and
an extension portion extending away from the second end of the body portion, wherein the trunnions extend laterally outwards from the body portion at or near the second end of the body portion.

9. The anchor of claim 8, wherein the body portion is generally cylindrical in shape.

10. The anchor of claim 8, wherein the body portion and the extension portion are interconnected by a breakaway connection which is configured to retain structural integrity under a first predetermined tensile load and to separate under a second predetermined tensile load which is greater than the first predetermined tensile load.

11. The anchor of claim 6, wherein:
the washer has an upper side, an opposed lower side, and a peripheral surface interconnecting the upper and lower sides; and
at least one of the upper and lower sides includes a relief configured to permit pivoting of the body portion to angles at the extreme ends of its range.

12. The anchor of claim 1 wherein:
the flange element is a cup having a concave seat; and
The housing includes an annular ball seat which is received in the concave seat of the cup.

13. The anchor of claim 12, wherein the housing includes:
a body portion extending between first and second ends; and
an extension portion extending away from the second end of the body portion, wherein the ball portion extends laterally outwards from the body portion at or near the second end of the body portion.

14. The anchor of claim 13, wherein the body portion and the extension portion are interconnected by a breakaway connection which is configured to retain structural integrity under a first predetermined tensile load and to separate under a second predetermined tensile load which is greater than the first predetermined tensile load.
